(19) United States
(12) Patent Application Publication
(10) Pub. No.: US 2014/0342913 A1
(43) Pub. Date: Nov. 20, 2014

(54) GENES PROVIDING TOLERANCE TO PDS INHIBITORS

(71) Applicant: SYNGENTA PARTICIPATIONS AG, Basel (CH)

(72) Inventors: Quideng Que, Durham, NC (US); John Luther Dawson, Durham, NC (US); Zhongying Chen, Durham, NC (US)

(73) Assignee: SYNGENTA PARTICIPATIONS AG, Basel (CH)

(21) Appl. No.: 14/169,370

(22) Filed: Jan. 31, 2014

Related U.S. Application Data
(60) Provisional application No. 61/823,941, filed on May 16, 2013.

(51) Int. Cl. C12N 15/82; C12N 9/02

(52) U.S. Cl. CPC C12N 15/8274; C12N 9/0069; C12Y 103/99031
USPC 504/237; 536/23.2; 435/320.1; 800/278; 800/300; 800/300.1

(57) ABSTRACT

The present invention relates to DNA molecules encoding a PDS inhibitor tolerant phytoene desaturease enzymes as well as constructs and plants comprising said enzymes. Also included are methods of using said enzymes, including use as a selectable marker, use to make transgenic plants resistant to PDS inhibitor herbicides and methods of controlling weeds.

### STATEMENT REGARDING ELECTRONIC SUBMISSION OF A SEQUENCE LISTING

A Sequence Listing in ASCII text format, submitted under 37 C.F.R. §1.821, entitled “80224-US-REG-ORG_NAT-1PDS_ST25.txt”, 28.2 KB bytes in size, generated on May 10, 2013 and filed via EFS-Web is provided in lieu of a paper copy. This Sequence Listing is hereby incorporated by reference into the specification for its disclosures.

### FIELD OF THE INVENTION

This invention relates to genes for providing herbicide resistance in plants and, more particularly, to phytoene dehydrogenase (or phytoene desaturase) genes derived from bacteria and fungi that provide resistance to PDS (phytoene desaturase) inhibitors, and variants thereof. Genes encoding these phytoene dehydrogenase proteins can be introduced into plants and selected in the presence of herbicides such as norflurazon and fluridone which inhibits endogenous plant phytoene desaturase or zeta-carotene dehydrogenase activity. The transgenic plants containing these new phytoene dehydrogenase genes are herbicide-tolerant. More specifically, the invention comprises DNA and protein compositions of PDS inhibitor-resistant PDS genes, methods of use in plant cell culture, crop breeding, generation of transgenic plants, controlling weeds at a locus comprising the transgenic plants and to the transgenic plants comprising the phytoene desaturase DNA.

### BACKGROUND OF THE INVENTION

Currently, there are 2 classes of genes involved in carotenoid biosynthesis that have been shown to confer resistance to herbicides. One class is crtP-type phytoene desaturases (PDS) from cyanobacteria, algae and plants. PDS converts phytoene to zeta-carotene (FIG. 1). Normally, these PDS proteins are sensitive to herbicides, but herbicide-resistant mutants of cyanobacterial or plant phytoene desaturases have been identified, such as Synechococcus PCC7942 PDS Val403Gly mutant or Hydrilla verticillata PDS Lys304Thr mutant (Chamovitz et al., 1991, Wagner et al., 2002, Arias et al., 2006, Albrecht et al., WO04007691).

Another class is bacterial Erwinia herbicola (also known as Pantoea stewartii) crtI which directly converts phytoene to lycopene or its downstream carotenoid compounds (FIG. 1) and is insensitive to plant PDS and ZDS inhibitors like norflurazon and fluridone (Misawa et al., 1993, Misawa et al., 1994). crtP-type PDS from cyanobactria, alga and plants are sensitive to herbicides. Their herbicide resistant mutants have good tolerance, but can be improved for trait development. The Erwinia crtI is not sensitive to several plant PDS or ZDS herbicides and is a good candidate gene for herbicide resistance trait. However, it was shown that expression of Erwinia crtI can alter xanthophyll metabolism which might not be desirable (Misawa et al., 1994). In bacteria and yeast, there are several other crtI-like enzymes that catalyze dehydrogenation of phytoene, but possessing different activities and producing different products than either plant-type (crt-P) PDS or Erwinia crtI (FIG. 1). Rhodobacter capsulatus crtI protein catalyzes a three-step desaturation of phytoene to form neurosporene, instead of lycopene as produced by Erwinia crtI. Also, there are other crtI homologs that only catalyze the 4-step desaturation step forming lycopene, such as crtI genes from Gloeobacter violaceous, Xanthophyllomyces dendrorhous and Rubrivivax These crtI genes may have advantage over Erwinia crtI in that they only catalyze formation of lycopene but not other metabolites. Neurospora al-1 gene encodes a crtI homolog which catalyzes a five-step desaturation of phytoene to form 3,4-didehydrolycopene (Sandman, 2009). Prior to the instant invention, these crtI genes have not been tested or used to engineer and optimize herbicide tolerance, such as co-expression with other crtI genes and mutant PDS genes.

There is a great need in plant molecular biology for a diversity of genes that can provide herbicide tolerance, both for use as a transgenic trait to enable control of weeds and to provide a selectable marker phenotype for use in plant transformation. Tolerant PDS enzymes are useful as selectable markers during transformation where PDS inhibitor selection can be used, i.e. where transformed plant cells comprising the tolerant PDS are selected due to their ability to survive on PDS inhibitor-containing media. The present invention provides DNA and protein compositions of PDS inhibitor tolerant enzymes. The present invention also provides DNA constructs useful in plants and transgenic plants that exhibit PDS inhibitor tolerance.

### SUMMARY OF THE INVENTION

In one aspect of the invention, there is provided an isolated DNA molecule encoding a modified PDS enzyme wherein said enzyme comprises an amino acid sequence selected from the group consisting of: SEQ ID NO. 1, SEQ ID NO. 2, SEQ ID NO. 3 and SEQ ID NO. 4.

In another aspect of the invention, there is provided an isolated DNA molecule encoding a modified PDS enzyme, wherein the DNA molecule is selected from the group consisting of: SEQ ID NO. 5, SEQ ID NO. 6, SEQ ID NO. 7 and SEQ ID NO. 8.

In another aspect of the invention, there is provided a DNA construct comprising a promoter that functions in plant cells operably linked to a fusion protein, said fusion protein comprising a chloroplast transit peptide fused to an isolated DNA molecule encoding a modified PDS enzyme wherein said enzyme comprises an amino acid sequence selected from the group consisting of: SEQ ID NO. 1, SEQ ID NO. 2, SEQ ID NO. 3 and SEQ ID NO. 4.

In another aspect of the invention, there is provided a method of preparing a PDS inhibitor tolerant plant comprising the steps of:
a. contacting a recipient plant cell with the DNA construct comprising an expression cassette of modified PDS gene, wherein said DNA construct is incorporated into the genome of the recipient plant cell;
b. regenerating the recipient plant cell into a plant; and
c. applying an effective dose of a PDS inhibitor to the plant, wherein the plant displays a PDS inhibitor tolerant phenotype.

In another aspect of the invention, there is provided a PDS inhibitor tolerant plant where in the plant comprises a DNA molecule encoding a modified PDS enzyme wherein said enzyme comprises an amino acid sequence selected from the group consisting of: SEQ ID NO. 1, SEQ ID NO. 2, SEQ ID NO. 3 and SEQ ID NO. 4.

In another aspect of the invention, there is provided a method of controlling weeds in a field of PDS inhibitor tolerant crop plants comprising applying to said field of PDS inhibitor tolerant crop plants an effective dose of a PDS inhibitor containing herbicide, wherein said PDS inhibitor tolerant crop plant contains a DNA construct comprising a promoter that functions in plant cells operably linked to a DNA molecule that encodes a chloroplast transit peptide linked to an isolated DNA molecule encoding a modified PDS enzyme wherein said enzyme comprises an amino acid sequence selected from the group consisting of: SEQ ID NO. 1, SEQ ID NO. 2, SEQ ID NO. 3 and SEQ ID NO. 4.

In another aspect of the invention, there is provided a method of plant transformation wherein the selectable marker is an enzyme encoded by an isolated DNA molecule encoding a modified PDS enzyme wherein said enzyme comprises an amino acid sequence selected from the group consisting of: SEQ ID NO. 1, SEQ ID NO. 2, SEQ ID NO. 3 and SEQ ID NO. 4, and the selection agent is a PDS inhibitor herbicide.

### BRIEF DESCRIPTION OF THE DRAWINGS

FIG. 1. Phytoene dehydrogenation (desaturation) steps catalyzed by desaturases from different organisms (Rhodobacter, Gleobacter, Erwinia and Neuropspora)

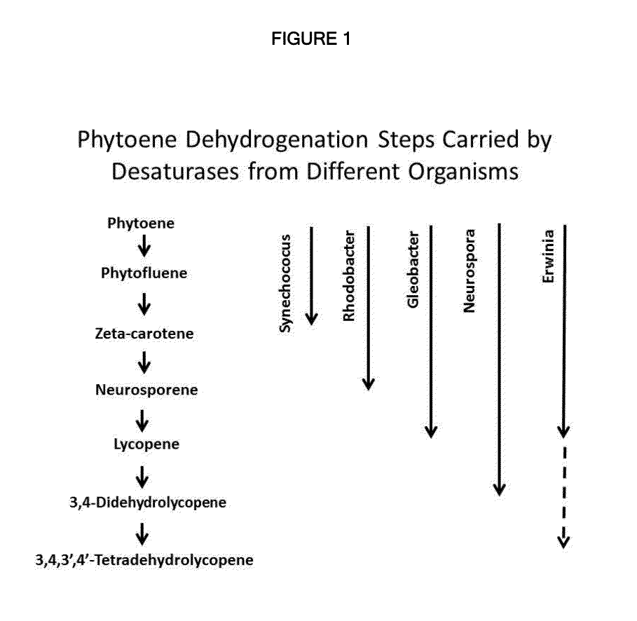

FIG. 2. Plasmid map of binary vector pBSC18238 containing an expression cassette comprising a gene encoding a fusion protein (cCrtIPt-01) of FNR CTP and Rhodobacter capsulatus CrtI

FIG. 3. Plasmid map of binary vector pBSC18239 containing an expression cassette comprising a gene encoding a fusion protein (cCrtIPt-02) of FNR CTP and Gloeobacter violaceous CrtI

FIG. 4. Plasmid map of binary vector pBSC18240 containing an expression cassette comprising a gene encoding a fusion protein (cCrtIPt-03) of FNR CTP and Neurospora crassa CrtI

FIG. 5. Plasmid map of binary vector pBSC18241 containing an expression cassette comprising a gene encoding a fusion protein (cCrtIPt-04) of FNR CTP and Synechococcus PCC7942 PDS V403G mutant

FIG. 6. Herbicide Solicam® (active ingredient: norflurazon) resistance phenotypes of T1 progeny from transgenic event SYHT093705C007A containing T-DNA of 18239 (Left: control, nontransgenic Jack plants; Right, transgenic SYHT093705C007A T1 plants segregating for transgene and resistance phenotypes;)

FIG. 7. Plasmid map of binary vector 18340 for plant transformation

FIG. 8. Plasmid map of binary vector 18341 for plant transformation

FIG. 9. Plasmid map of binary vector 18900 for plant transformation

FIG. 10. Plasmid map of binary vector 18932 for plant transformation

### LIST OF SEQUENCES

SEQ ID NO. 1: Amino acid sequence of Rhodobacter capsulatus crtI protein

SEQ ID NO. 2: Amino acid sequence of Gloeobacter violaceous crtI protein

SEQ ID NO. 3: Amino acid sequence of Neurospora crassa crtI protein

SEQ ID NO. 4: Amino acid sequence of Synechococcus PCC7942 mutant protein

SEQ ID NO. 5: DNA sequence of Rhodobacter capsulatus crtI

SEQ ID NO. 6: DNA sequence of Gloeobacter violaceous crtI

SEQ ID NO. 7: DNA sequence of Neurospora crassa crtI

SEQ ID NO. 8: DNA sequence of Synechococcus PCC7942 mutant

SEQ ID NO. 9: xFNR amino acid sequence

SEQ ID NO. 10: xFNR DNA sequence

### DETAILED DESCRIPTION OF THE INVENTION

The present invention is drawn to compositions and methods for regulating herbicide resistance in organisms, particularly in plants or plant cells. The methods involve transforming organisms with nucleotide sequences encoding the PDS inhibitor resistant genes of the invention. The nucleotide sequences of the invention are useful for preparing plants that show increased tolerance to the PDS inhibitor herbicides and for use as selectable markers in transformation. Thus, transformed bacteria, plants, plant cells, plant tissues and seeds are provided. Compositions include nucleic acids and proteins relating to herbicide tolerance in microorganisms and plants as well as transformed bacteria, plants, plant tissues and seeds. Nucleotide sequences of the PDS genes and the amino acid sequences of the proteins encoded thereby are disclosed. The sequences find use in the construction of expression vectors for subsequent transformation into plants of interest, as selectable markers, and the like.

The PDS enzyme functions in plant chloroplasts, therefore, chloroplast transit peptides (CTP) are engineered in a DNA molecule to encode a fusion of the CTP to the N terminus of a PDS enzyme, creating a chimeric molecule. A chimeric polynucleic acid coding sequence is comprised of two or more open reading frames joined in-frame that encode a chimeric protein, for example, a chloroplast transit peptide and a PDS enzyme. A chimeric gene refers to the multiple genetic elements derived from heterologous sources operably linked to comprise a gene. The CTP directs the resistant enzyme into the plant chloroplast. The CTP is cleaved from the nzyme at the chloroplast membrane to create a mature PDS enzyme that refers to the polypeptide sequence of the processed protein product remaining after the chloroplast transit peptide has been removed.

The native CTP may be substituted with a heterologous CTP during construction of a transgene plant expression cassette. Many chloroplast-localized proteins, including PDS, are expressed from nuclear genes as precursors and are targeted to the chloroplast by a chloroplast transit peptide (CTP) that is removed during the import steps. Examples of other such chloroplast proteins include EPSPS, the small subunit (SSU) of ribulose-1,5,-bisphosphate carboxylase (rubisco), ferredoxin, ferredoxin oxidoreductase, the light-harvesting complex protein I and protein II, and thioredoxin F. It has been demonstrated in vivo and in vitro that non-chloroplast proteins may be targeted to the chloroplast by use of protein fusions with a CTP and that a CTP sequence is sufficient to target a protein to the chloroplast. Incorporation of a suitable chloroplast transit peptide, such as, the Arabidopsis thaliana EPSPS CTP (Klee et al., Mol. Gen. Genet. 210:437-442 (1987), and the Petunia hybrid EPSPS CTP (Della-Cioppa et al., Proc. Natl. Acad. Sci. USA 83:6873-6877 (1986) has been shown to target heterologous EPSPS protein to chloroplasts in transgenic plants. The production of glyphosate tolerant plants by expression of a fusion protein comprising an amino-terminal CTP with a glyphosate resistant EPSPS enzyme is well known by those skilled in the art, (U.S. Pat. No. 5,627,061, U.S. Pat. No. 5,633,435, U.S. Pat. No. 5,312,910, EP 0218571, EP 189707, EP 508909, and EP 924299). Those skilled in the art will recognize that various chimeric constructs can be made that utilize the functionality of a particular CTP to import PDS enzymes into the plant cell chloroplast.

Modification and changes may be made in the structure of the DNA polynucleotides of the invention and still obtain a DNA molecule that transcribes an mRNA that encodes the modified functional PDS protein of the present invention. Amino-acid substitutions or amino-acid variants, are preferably substitutions of a single amino-acid residue for another amino-acid residue at one or more positions within the protein. Substitutions, deletions, insertions or any combination thereof can be combined to arrive at a final construct.

It is known that the genetic code is degenerate. The amino acids and their RNA codon(s) are listed below in Table 1.

TABLE 1
Amino acids and the RNA codons that encode

| Amino Acid | |
|---|---|
| Full name; 3 letter | |
| code; 1 letter code | Codons |
| Alanine; Ala; A | GCA GCC GCG GCU |
| Cysteine; Cys; C | UGC UGU |
| Aspartic acid; Asp; D | GAC GAU |
| Glutamic acid; Glu; E | GAA GAG |
| Phenylalanine; Phe; F | UUC UUU |
| Glycine; Gly; G | GGA GGC GGG GGU |
| Histidine; His; H | CAC CAU |
| Isoleucine; Ile; I | AUA AUC AUU |
| Lysine; Lys; K | AAA AAG |
| Leucine; Leu; L | UUA UUG CUA CUC CUG CUU |
| Methionine; Met; M | AUG |
| Asparagine; Asn; N | AAC AAU |
| Proline; Pro; P | CCA CCC CCG CCU |
| Glutamine; Gln; Q | CAA CAG |
| Arginine; Arg; R | AGA AGG CGA CGC CGG CGU |
| Serine; Ser; S | AGC AGU UCA UCC UCG UCU |
| Threonine; Thr; T | ACA ACC ACG ACU |
| Valine; Val; V | GUA GUC GUG GUU |
| Tryptophan; Trp; W | UGG |
| Tyrosine; Tyr; Y | UAC UAU |

The codons are described in terms of RNA bases, for example adenine, uracil, guanine and cytosine, it is the mRNA that is directly translated into polypeptides. It is understood that when designing a DNA polynucleotide for use in a construct, the DNA bases would be substituted, for example, thymine instead of uracil. Codon refers to a sequence of three nucleotides that specify a particular amino acid. Codon usage or “codon bias” refers to the frequency of use of codons encoding amino acids in the coding sequences of organisms. A codon usage table would be consulted when selecting substituting codons for an artificial DNA sequence. The sequence of codons provides a coding sequence that refers to the region of continuous sequential nucleic acid triplets encoding a protein, polypeptide, or peptide sequence. The term “encoding DNA” refers to chromosomal DNA, plasmid DNA, cDNA, or artificial DNA polynucleotide that encodes any of the proteins discussed herein. “Plasmid” refers to a circular, extrachromosomal, self-replicating piece of DNA.

The term “endogenous” refers to materials originating from within an organism or cell. “Exogenous” refers to materials originating from outside of an organism or cell. This typically applies to nucleic acid molecules used in producing transformed or transgenic host cells and plants.

The term “genome” as it applies to bacteria encompasses both the chromosome and plasmids within a bacterial host cell. Encoding nucleic acids of the present invention introduced into bacterial host cells can therefore be either chromosomally-integrated or plasmid-localized. The term “genome” as it applies to plant cells encompasses not only chromosomal DNA found within the nucleus, but organelle DNA found within subcellular components of the cell. The term “gene” refers to polynucleic acids that comprise chromosomal DNA, plasmid DNA, cDNA, an artificial DNA polynucleotide, or other DNA that is transcribed into an RNA molecule, wherein the RNA may encode a peptide, polypeptide, or protein, and the genetic elements flanking the coding sequence that are involved in the regulation of expression of the mRNA or polypeptide of the present invention. A “fragment” of a gene is a portion of a full-length polynucleic acid molecule that is of at least a minimum length capable of transcription into a RNA, translation into a peptide, or useful as a probe or primer in a DNA detection method.

Polynucleic acids of the present invention introduced into plant cells can therefore be either chromosomally-integrated or organelle-localized. The PDSs of the present invention are targeted to the chloroplast by a chloroplast transit peptide located at the N-terminus of the coding sequence. Alternatively, the gene encoding the PDSs may be integrated into the chloroplast genome, thereby eliminating the need for a chloroplast transit peptide.

“Heterologous DNA” sequence refers to a polynucleotide sequence that originates from a foreign source or species or, if from the same source, is modified from its original form. “Homologous DNA” refers to DNA from the same source as that of the recipient cell.

“Hybridization” refers to the ability of a strand of nucleic acid to join with a complementary strand via base pairing. Hybridization occurs when complementary sequences in the two nucleic acid strands bind to one another. The nucleic acid probes and primers of the present invention hybridize under stringent conditions to a target DNA sequence. Any conventional nucleic acid hybridization or amplification method can be used to identify the presence of DNA from a transgenic event in a sample. A transgenic “event” is produced by transformation of a plant cell with heterologous DNA, i.e., a nucleic acid construct that includes a transgene of interest; regeneration of a population of plants resulting from the insertion of the transgene into the genome of the plant cell, and selection of a particular plant characterized by insertion into a particular genome location. The term “event” refers to the original transformant plant and progeny of the transformant that include the heterologous DNA. The term “event” also includes progeny produced by a sexual outcross between the event and another plant that wherein the progeny includes the heterologous DNA. Nucleic acid molecules or fragments thereof are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., Molecular Cloning—A Laboratory Manual, 2nd. ed., Cold Spring Harbor Laboratory, Cold Spring Harbor, N.Y. (1989), herein referred to as Sambrook et al., (1989), and by Haymes et al., In: Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

As used herein, a substantially homologous sequence is a nucleic acid sequence that will specifically hybridize to the complement of the nucleic acid sequence to which it is being compared under high stringency conditions. The term “stringent conditions” is functionally defined with regard to the hybridization of a nucleic-acid probe to a target nucleic acid (such as, to a particular nucleic-acid sequence of interest) by the specific hybridization procedure discussed in Sambrook et al., 1989, at 9.52-9.55. See also, Sambrook et al., 1989 at 9.47-9.52, 9.56-9.58; Kanehisa, (Nucl. Acids Res. 12:203-213, 1984); and Wetmur and Davidson, (J. Mol. Biol. 31:349-370, 1988). Accordingly, the nucleotide sequences of the invention may be used for their ability to selectively form duplex molecules with complementary stretches of DNA fragments. Depending on the application envisioned, one will desire to employ varying conditions of hybridization to achieve varying degrees of selectivity of probe towards target sequence. For applications requiring high selectivity, one will typically desire to employ relatively stringent conditions to form the hybrids, for example, one will select relatively low salt and/or high temperature conditions, such as provided by about 0.02 M to about 0.15 M NaCl at temperatures of about 50° C. to about 70° C. A stringent condition, for example, is to wash the hybridization filter at least twice with high-stringency wash buffer (0.2×SSC, 0.1% SDS, 65° C.). Appropriate stringency conditions that promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. Such selective conditions tolerate little, if any, mismatch between the probe and the template or target strand. Detection of DNA molecules via hybridization is well known to those of skill in the art, and the teachings of U.S. Pat. Nos. 4,965,188 and 5,176,995 are exemplary of the methods of hybridization analyses.

“Identity” refers to the degree of similarity between two polynucleic acid or protein sequences. An alignment of the two sequences is performed by a suitable computer program. A widely used and accepted computer program for performing sequence alignments is CLUSTALW v1.6 (Thompson, et al. Nucl. Acids Res., 22: 4673-4680, 1994). The number of matching bases or amino acids is divided by the total number of bases or amino acids, and multiplied by 100 to obtain a percent identity. For example, if two 580 base pair sequences had 145 matched bases, they would be 25 percent identical. If the two compared sequences are of different lengths, the number of matches is divided by the shorter of the two lengths. For example, if there are 100 matched amino acids between 200 and 400 amino acid proteins, they are 50 percent identical with respect to the shorter sequence. If the shorter sequence is less than 150 bases or 50 amino acids in length, the number of matches are divided by 150 (for nucleic acid bases) or 50 (for amino acids), and multiplied by 100 to obtain a percent identity.

“Intron” refers to a genetic element that is a portion of a gene not translated into protein, even though it is transcribed into RNA, the intron sequence being “spliced out” from the mature messenger RNA.

An “isolated” nucleic acid molecule is substantially separated away from other nucleic acid sequences with which the nucleic acid is normally associated, such as, from the chromosomal or extrachromosomal DNA of a cell in which the nucleic acid naturally occurs. A nucleic acid molecule is an isolated nucleic acid molecule when it comprises a transgene or part of a transgene present in the genome of another organism. The term also embraces nucleic acids that are biochemically purified so as to substantially remove contaminating nucleic acids and other cellular components. The term “transgene” refers to any polynucleic acid molecule normative to a cell or organism transformed into the cell or organism. “Transgene” also encompasses the component parts of a native plant gene modified by insertion of a normative polynucleic acid molecule by directed recombination or site specific mutation.

“Isolated,” “Purified,” “Homogeneous” polypeptides. A polypeptide is “isolated” if it has been separated from the cellular components (nucleic acids, lipids, carbohydrates, and other polypeptides) that naturally accompany it or that is chemically synthesized or recombinant. A polypeptide molecule is an isolated polypeptide molecule when it is expressed from a transgene in another organism. A monomeric polypeptide is isolated when at least 60% by weight of a sample is composed of the polypeptide, preferably 90% or more, more preferably 95% or more, and most preferably more than 99%. Protein purity or homogeneity is indicated, for example, by polyacrylamide gel electrophoresis of a protein sample, followed by visualization of a single polypeptide band upon staining the polyacrylamide gel; high pressure liquid chromatography; or other conventional methods. Proteins can be purified by any of the means known in the art, for example as described in Guide to Protein Purification, ed. Deutscher, Meth. Enzymol. 185, Academic Press, San Diego, 1990; and Scopes, Protein Purification: Principles and Practice, Springer Verlag, New York, 1982.

A first nucleic-acid molecule is “operably linked” with a second nucleic-acid molecule when the first nucleic-acid molecule is placed in a functional relationship with the second nucleic-acid molecule. For example, a promoter is operably linked to a protein-coding nucleic acid sequence if the promoter effects the transcription or expression of the coding sequence. Generally, operably linked DNA molecules are contiguous and, where necessary to join two protein-coding regions, in reading frame.

The term “plant” encompasses any higher plant and progeny thereof, including monocots (for example, corn, rice, wheat, barley, etc.), dicots (for example, soybean, cotton, canola, tomato, potato, Arabidopsis, tobacco, etc.), gymnosperms (pines, firs, cedars, etc.) and includes parts of plants, including reproductive units of a plant (for example, seeds, bulbs, tubers, fruit, flowers, etc.) or other parts or tissues from that the plant can be reproduced.

“Polyadenylation signal” or “polyA signal” refers to a nucleic acid sequence located 3′ to a coding region that causes the addition of adenylate nucleotides to the 3′ end of the mRNA transcribed from the coding region.

“Polymerase chain reaction (PCR)” refers to a DNA amplification method that uses an enzymatic technique to create multiple copies of one sequence of nucleic acid (amplicon). Copies of a DNA molecule are prepared by shuttling a DNA polymerase between two amplimers. The basis of this amplification method is multiple cycles of temperature changes to denature, then re-anneal amplimers (DNA primer molecules), followed by extension to synthesize new DNA strands in the region located between the flanking amplimers. Nucleic-acid amplification can be accomplished by any of the various nucleic-acid amplification methods known in the art, including the polymerase chain reaction (PCR). A variety of amplification methods are known in the art and are described, inter alia, in U.S. Pat. Nos. 4,683,195 and 4,683,202 and in PCR Protocols: A Guide to Methods and Applications, ed. Innis et al., Academic Press, San Diego, 1990. PCR amplification methods have been developed to amplify up to 22 kb of genomic DNA and up to 42 kb of bacteriophage DNA (Cheng et al., Proc. Natl. Acad. Sci. USA 91:5695-5699, 1994). These methods as well as other methods known in the art of DNA amplification may be used in the practice of the present invention.

The term “promoter” or “promoter region” refers to a polynucleic acid molecule that functions as a regulatory element, usually found upstream (5′) to a coding sequence, that controls expression of the coding sequence by controlling production of messenger RNA (mRNA) by providing the recognition site for RNA polymerase and/or other factors necessary for start of transcription at the correct site. As contemplated herein, a promoter or promoter region includes variations of promoters derived by means of ligation to various regulatory sequences, random or controlled mutagenesis, and addition or duplication of enhancer sequences. The promoter region disclosed herein, and biologically functional equivalents thereof, are responsible for driving the transcription of coding sequences under their control when introduced into a host as part of a suitable recombinant DNA construct, as demonstrated by its ability to produce mRNA.

A “recombinant” nucleic acid is made by a combination of two otherwise separated segments of nucleic acid sequence, for example, by chemical synthesis or by the manipulation of isolated segments of polynucleic acids by genetic engineering techniques. The term “recombinant DNA construct” refers to any agent such as a plasmid, cosmid, virus, autonomously replicating sequence, phage, or linear or circular single-stranded or double-stranded DNA or RNA nucleotide sequence, derived from any source, capable of genomic integration or autonomous replication, comprising a DNA molecule that one or more DNA sequences have been linked in a functionally operative manner. Such recombinant DNA constructs are capable of introducing a 5′ regulatory sequence or promoter region and a DNA sequence for a selected gene product into a cell in such a manner that the DNA sequence is transcribed into a functional mRNA that is translated and therefore expressed. Recombinant DNA constructs may be constructed to be capable of expressing antisense RNAs, or stabilized double stranded antisense RNA in order to inhibit expression of a specific target RNA of interest.

“Resistance” refers to an enzyme that is able to function in the presence of a toxin, for example, PDS enzymes having catalytic activity that is unaffected by a herbicide concentration that normally disrupts the same activity in the wild type enzyme, for example, the PDS enzymes of the present invention. An enzyme that has resistance to an herbicide may also have the function of detoxifying the herbicide, for example, phosphinothricin acetyltransferase, and glyphosate oxidoreductase.

“Selectable marker” refers to a polynucleic acid molecule that encodes a protein, which confers a phenotype facilitating identification of cells containing the polynucleic acid molecule. Selectable markers include those genes that confer resistance to antibiotics (for example, ampicillin, kanamycin), complement a nutritional deficiency (for example, uracil, histidine, leucine), or impart a visually distinguishing characteristic (for example, color changes or fluorescence). Useful dominant selectable marker genes include genes encoding antibiotic resistance genes (for example, neomycin phosphotransferase, npt); and herbicide resistance genes (for example, phosphinothricin acetyltransferase, class II EPSPSs and class III EPSPSs, modified class I EPSPSs). A useful strategy for selection of transformants for herbicide resistance is described, for example, in Vasil, Cell Culture and Somatic Cell Genetics of Plants, Vols. I-III, Laboratory Procedures and Their Applications Academic Press, New York (1984).

An “artificial polynucleotide” as used in the present invention is a DNA sequence designed according to the methods of the present invention and created as an isolated DNA molecule for use in a DNA construct that provides expression of a protein in host cells, or for the purposes of cloning into appropriate constructs or other uses known to those skilled in the art. Computer programs are available for these purposes, including but not limited to the “BestFit” or “Gap” programs of the Sequence Analysis Software Package, Genetics Computer Group (GCG), Inc., University of Wisconsin Biotechnology Center, Madison, Wis. 53711. The artificial polynucleotide may be created by a one or more methods known in the art, that include, but are not limited to: overlapping PCR. An artificial polynucleotide as used herein, is non-naturally occurring and can be substantially divergent from other polynucleotides that code for the identical or nearly identical protein.

### Expression of a PDS Coding Sequence in Plants

DNA constructs are made that contain various genetic elements necessary for the expression of the PDS coding sequence in plants. “DNA construct” refers to the heterologous genetic elements operably linked to each other making up a recombinant DNA molecule and may comprise elements that provide expression of a DNA polynucleotide molecule in a host cell and elements that provide maintenance of the construct in the host cell. A plant expression cassette comprises the operable linkage of genetic elements that when transferred into a plant cell provides expression of a desirable gene product. “Plant expression cassette” refers to chimeric DNA segments comprising the regulatory elements that are operably linked to provide the expression of a transgene product in plants. Promoters, leaders, introns, transit peptide encoding polynucleic acids, 3′ transcriptional termination regions are all genetic elements that may be operably linked by those skilled in the art of plant molecular biology to provide a desirable level of expression or functionality to a PDS inhibitor resistant enzyme of the present invention. A DNA construct can contain one or more plant expression cassettes expressing the DNA molecules of the present invention or other DNA molecules useful in the genetic engineering of crop plants.

A variety of promoters specifically active in vegetative tissues, such as leaves, stems, roots and tubers, can be used to express the PDS polynucleic acid molecules of the present invention. Examples of tuber-specific promoters include, but are not limited to the class I and II patatin promoters (Bevan et al., EMBO J. 8:1899-1906, 1986; Koster-Topfer et al., Mol Gen Genet. 219:390-396, 1989; Mignery et al., Gene. 62:27-44, 1988; Jefferson et al., Plant Mol. Biol. 14: 995-1006, 1990), the promoter for the potato tuber ADPGPP genes, both the large and small subunits; the sucrose synthase promoter (Salanoubat and Belliard, Gene. 60:47-56, 1987; Salanoubat and Belliard, Gene 84: 181-185, 1989); and the promoter for the major tuber proteins including the 22 kd protein complexes and proteinase inhibitors (Hannapel, Plant Physiol. 101:703-704, 1993). Examples of leaf-specific promoters include, but are not limited to the ribulose biphosphate carboxylase (RBCS or RuBISCO) promoters (see, for example, Matsuoka et al., Plant J. 6:311-319, 1994); the light harvesting chlorophyll a/b binding protein gene promoter (see, for example, Shiina et al., Plant Physiol. 115:477-483, 1997; Casal et al., Plant Physiol. 116:1533-1538, 1998); and the Arabidopsis thaliana myb-related gene promoter (Atmyb5) (Li et al., FEBS Lett. 379:117-121, 1996). Examples of root-specific promoter include, but are not limited to the promoter for the acid chitinase gene (Samac et al., Plant Mol. Biol. 25:587-596, 1994); the root specific subdomains of the CaMV35S promoter that have been identified (Lam et al., Proc. Natl. Acad. Sci. (U.S.A.) 86:7890-7894, 1989); the ORF13 promoter from Agrobacterium rhizogenes that exhibits high activity in roots (Hansen et al., Mol. Gen. Genet. 254:337-343 (1997); the promoter for the tobacco root-specific gene TobRB7 (Yamamoto et al., Plant Cell 3:371-382, 1991); and the root cell specific promoters reported by Conkling et al. (Conkling et al., Plant Physiol. 93:1203-1211, 1990).

Another class of useful vegetative tissue-specific promoters is meristematic (root tip and shoot apex) promoters. For example, the “SHOOTMERISTEMLESS” and “SCARECROW” promoters, which are active in the developing shoot or root apical meristems can be used (Di Laurenzio et al., Cell 86:423-433, 1996; Long, Nature 379:66-69, 1996). Another example of a useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, for example, Enjuto et al., Plant Cell. 7:517-527, 1995). Also another example of a useful promoter is that which controls the expression of knI-related genes from maize and other species that show meristem-specific expression (see, for example, Granger et al., Plant Mol. Biol. 31:373-378, 1996; Kerstetter et al., Plant Cell 6:1877-1887, 1994; Hake et al., Philos. Trans. R. Soc. Lond. B. Biol. Sci. 350:45-51, 1995). Another example of a meristematic promoter is the Arabidopsis thaliana KNAT1 promoter. In the shoot apex, KNAT1 transcript is localized primarily to the shoot apical meristem; the expression of KNAT1 in the shoot meristem decreases during the floral transition and is restricted to the cortex of the inflorescence stem (see, for example, Lincoln et al., Plant Cell 6:1859-1876, 1994).

Suitable seed-specific promoters can be derived from the following genes: MAC1 from maize (Sheridan et al., Genetics 142:1009-1020, 1996; Cat3 from maize (GenBank No. L05934, Abler et al., Plant Mol. Biol. 22:10131-1038, 1993); viviparous-1 from Arabidopsis (Genbank No. U93215); Atimycl from Arabidopsis (Urao et al., Plant Mol. Biol. 32:571-57, 1996; Conceicao et al., Plant 5:493-505, 1994); napA from Brassica napus (GenBank No. J02798); the napin gene family from Brassica napus (Sjodahl et al., Planta 197:264-271, 1995, and others (Chen et al., Proc. Natl. Acad. Sci. 83:8560-8564, 1986).

The ovule-specific promoter for BEL1 gene can also be used (Reiser et al. Cell 83:735-742, 1995, GenBank No. U39944; Ray et al, Proc. Natl. Acad. Sci. USA 91:5761-5765, 1994). The egg and central cell specific MEA (FIS1) and FIS2 promoters are also useful reproductive tissue-specific promoters (Luo et al., Proc. Natl. Acad. Sci. USA, 97:10637-10642, 2000; Vielle-Calzada, et al., Genes Dev. 13:2971-2982, 1999).

A maize pollen-specific promoter has been identified in maize (Guerrero et al., Mol. Gen. Genet. 224:161-168, 1990). Other genes specifically expressed in pollen have been described (see, for example, Wakeley et al., Plant Mol. Biol. 37:187-192, 1998; Ficker et al., Mol. Gen. Genet. 257:132-142, 1998; Kulikauskas et al., Plant Mol. Biol. 34:809-814, 1997; Treacy et al., Plant Mol. Biol. 34:603-611, 1997).

It is recognized that additional promoters that may be utilized are described, for example, in U.S. Pat. Nos. 5,378,619, 5,391,725, 5,428,147, 5,447,858, 5,608,144, 5,608,144, 5,614,399, 5,633,441, 5,633,435, and 4,633,436. It is further recognized that the exact boundaries of regulatory sequences may not be completely defined, DNA fragments of different lengths may have identical promoter activity.

The translation leader sequence means a DNA molecule located between the promoter of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences include maize and petunia heat shock protein leaders, plant virus coat protein leaders, plant rubisco gene leaders among others (Turner and Foster, Molecular Biotechnology 3:225, 1995).

The “3′ non-translated sequences” means DNA sequences located downstream of a structural polynucleotide sequence and include sequences encoding polyadenylation and other regulatory signals capable of affecting mRNA processing or gene expression. The polyadenylation signal functions in plants to cause the addition of polyadenylate nucleotides to the 3′ end of the mRNA precursor. The polyadenylation sequence can be derived from the natural gene, from a variety of plant genes, or from T-DNA. An example of the polyadenylation sequence is the nopaline synthase 3′ sequence (nos 3′; Fraley et al., Proc. Natl. Acad. Sci. USA 80: 4803-4807, 1983). The use of different 3′ non-translated sequences is exemplified by Ingelbrecht et al., Plant Cell 1:671-680, 1989.

The laboratory procedures in recombinant DNA technology used herein are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Sambrook et al. (1989).

The DNA construct of the present invention may be introduced into the genome of a desired plant host by a variety of conventional transformation techniques that are well known to those skilled in the art. “Transformation” refers to a process of introducing an exogenous polynucleic acid molecule (for example, a DNA construct, a recombinant polynucleic acid molecule) into a cell or protoplast and that exogenous polynucleic acid molecule is incorporated into a host cell genome or an organelle genome (for example, chloroplast or mitochondria) or is capable of autonomous replication. “Transformed” or “transgenic” refers to a cell, tissue, organ, or organism into which a foreign polynucleic acid, such as a DNA vector or recombinant polynucleic acid molecule. A “transgenic” or “transformed” cell or organism also includes progeny of the cell or organism and progeny produced from a breeding program employing such a “transgenic” plant as a parent in a cross and exhibiting an altered phenotype resulting from the presence of the foreign polynucleic acid molecule.

Methods of transformation of plant cells or tissues include, but are not limited to Agrobacterium mediated transformation method and the Biolistics or particle-gun mediated transformation method. Suitable plant transformation vectors for the purpose of Agrobacterium mediated transformation include-those elements derived from a tumor inducing (Ti) plasmid of Agrobacterium tumefaciens, for example, right border (RB) regions and left border (LB) regions, and others disclosed by Herrera-Estrella et al., Nature 303:209 (1983); Bevan, Nucleic Acids Res. 12:8711-8721 (1984); Klee et al., Bio-Technology 3(7):637-642 (1985). In addition to plant transformation vectors derived from the Ti or root-inducing (Ri) plasmids of Agrobacterium, alternative methods can be used to insert the DNA constructs of this invention into plant cells. Such methods may involve, but are not limited to, for example, the use of liposomes, electroporation, chemicals that increase free DNA uptake, free DNA delivery via microprojectile bombardment, and transformation using viruses or pollen.

DNA constructs can be prepared that incorporate the PDS coding sequences of the present invention for use in directing the expression of the sequences directly from the host plant cell plastid. Examples of such constructs suitable for this purpose and methods that are known in the art and are generally described, for example, in Svab et al., Proc. Natl. Acad. Sci. USA 87:8526-8530, (1990) and Svab et al., Proc. Natl. Acad. Sci. USA 90:913-917 (1993) and in U.S. Pat. No. 5,693,507. It is contemplated that plastid transformation and expression of PDS enzymes of the present invention will provide PDS inhibitor tolerance to the plant cell.

When adequate numbers of cells containing the exogenous polynucleic acid molecule encoding polypeptides from the present invention are obtained, the cells can be cultured, then regenerated into whole plants. “Regeneration” refers to the process of growing a plant from a plant cell (for example, plant protoplast or explant). Such regeneration techniques rely on manipulation of certain phytohormones in a tissue culture growth medium, typically relying on a biocide and/or herbicide marker that has been introduced together with the desired nucleotide sequences. Choice of methodology for the regeneration step is not critical, with suitable protocols being available for hosts from Leguminoseae (for example, alfalfa, soybean, clover), Umbelliferae (carrot, celery, parsnip), Cruciferae (for example, cabbage, radish, canola/rapeseed), Cucurbitaceae (for example, melons and cucumber), Gramineae (for example, wheat, barley, rice, maize), Solanaceae (for example, potato, tobacco, tomato, peppers), various floral crops, such as sunflower, and nut-bearing trees, such as almonds, cashews, walnuts, and pecans. See, for example, Ammirato et al., Handbook of Plant Cell Culture—Crop Species. Macmillan Publ. Co. (1984); Shimamoto et al., Nature 338:274-276 (1989); Fromm, UCLA Symposium on Molecular Strategies for Crop Improvement, Apr. 16-22, 1990. Keystone, Colo. (1990); Vasil et al., Bio/Technology 8:429-434 (1990); Vasil et al., Bio/Technology 10:667-674 (1992); Hayashimoto, Plant Physiol. 93:857-863 (1990); and Datta et al., Bio-technology 8:736-740 (1990). Such regeneration techniques are described generally in Klee et al., Ann. Rev. Plant Phys. 38:467-486 (1987).

The development or regeneration of transgenic plants containing the exogenous polynucleic acid molecule that encodes a polypeptide of interest is well known in the art. Preferably, the regenerated plants are self-pollinated to provide homozygous transgenic plants, as discussed above. Otherwise, pollen obtained from the regenerated plants is crossed to seed-grown plants of agronomically important lines. Conversely, pollen from plants of these important lines is used to pollinate regenerated plants.

Plants that can be made to have enhanced PDS inhibitor tolerance by practice of the present invention include, but are not limited to, Acacia, alfalfa, aneth, apple, apricot, artichoke, arugula, asparagus, avocado, banana, barley, beans, beet, blackberry, blueberry, broccoli, brussels sprouts, cabbage, canola, cantaloupe, carrot, cassava, cauliflower, celery, cherry, cilantro, citrus, clementines, coffee, corn, cotton, cucumber, Douglas fir, eggplant, endive, escarole, eucalyptus, fennel, figs, forest trees, gourd, grape, grapefruit, honey dew, jicama, kiwifruit, lettuce, leeks, lemon, lime, Loblolly pine, mango, melon, mushroom, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, raspberry, rice, rye, sorghum, Southern pine, soybean, spinach, squash, strawberry, sugarbeet, sugarcane, sunflower, sweet potato, sweetgum, tangerine, tea, tobacco, tomato, turf, a vine, watermelon, wheat, yams, and zucchini.

Herein, examples are provided that show phytoene desaturase genes from several sources, including Rhodobacter, Gloeobacter, Neurospora and Synechococcus PCC7942, used to engineer and optimize herbicide tolerance. Specifically, the crtI and PDS protein sequences are fused with a plastid transit peptide such as FNR or Rubisco transit peptide at the N-terminus and the protein sequences are back-translated into DNA sequences for DNA synthesis. These fusion protein coding sequences are then placed under control of a plant promoter such as Cestrum promoter (CMP) to express these phytoene dehydrogenase activities in plant tissue. The DNA vectors are then transformed into plant cells to generate transgenic plants containing expression cassette of Rhodobacter and Gloeobacter crtI fusion proteins. Plant are selected with carotenoid biosynthesis inhibitors that inhibit plant phytoene desaturase (PDS) like norflurazon, floridone, fluridone, diflufenican, flurtamone, flurochloridone and picolinafen, and/or Zeta-carotene desaturase (ZDS) inhibitor, LS-80707, SAN 380H, RH1965, and Ref.31 (Hamprecht G. and Witschel M., 2007 Herbicides with Bleaching Properties. In: Modern Crop Protection Compounds, edited by W Kramer and U Schirmer. Wiley-VCH Verlag GmbH and Co.). Transgenic plants can be developed into herbicide tolerance trait against PDS or ZDS inhibitors.

These examples are provided to better elucidate the practice of the present invention and should not be interpreted in any way to limit the scope of the present invention. Those skilled in the art will recognize that various modifications, additions, substitutions, truncations, etc., can be made to the methods and genes described herein while not departing from the spirit and scope of the present invention.

### EXAMPLES

### Example 1

### Synthesis of Genes Encoding Phytoene Dehydrogenases (Desaturases)

Phytoene dehydrogenases (desaturases) from Rhodobacter capsulatus, Gloeobacter violaceous, Neurospora crassa and Synechococcus PCC7942 were fused with the cyanelle targeting transit peptide of ferredoxin-NADP+ reductase (xFNR) from Cyanophora paradoxa to generate herbicide tolerant fusion proteins. (SEQ ID NOs. 1-4 fused with SEQ ID NO. 9). xFNR transit peptide served as the plastid targeting sequence in the transformed plant cells. Functional transit peptides derived from higher plant proteins such as Rubsico, HPPD, ALS and EPSPS can be used to replace xFNR to target the crtI/PDS proteins into the plastid.

The protein sequences for each dehydrogenase were back-translated into DNA coding sequences with soybean optimized codons (SEQ ID NOs 5-8 and SEQ ID NO. 10 (xFNR)). To facilitate cloning the following were performed for each sequence: a BamHI restriction site was added to the 5′ end followed by a Kozak sequence TAAACC (5′-GGATCCTAAACC-3′); a SacI site was added to the 3′-end after the STOP codon; a NcoI site was added at the start of translation CCATGG where ATG is the start codon; all internal BamHI, SacI, SanDI, RsrII, NcoI sites were removed. DNA sequences encoding different fusion proteins were synthesized through a contract service provider and cloned into cloning vector as BamHI-SacI fragments.

### Example 2

### Construction of Plant Expression and Transformation Vectors

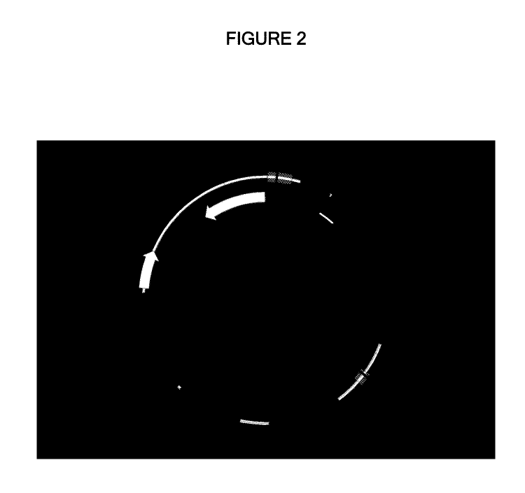

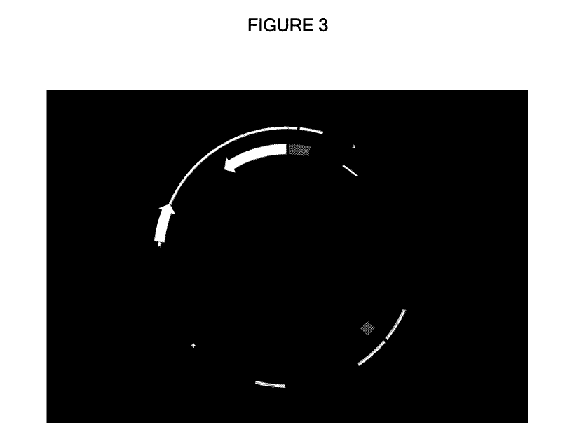

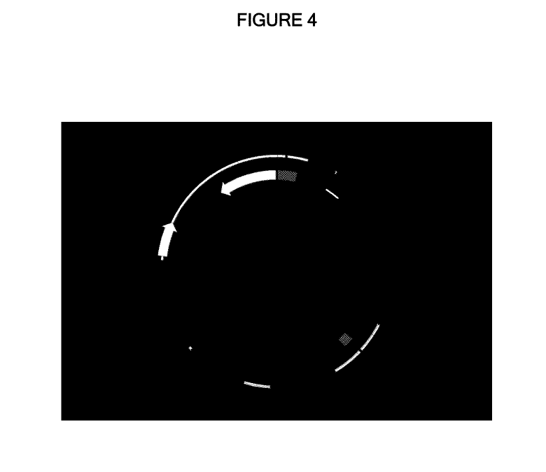

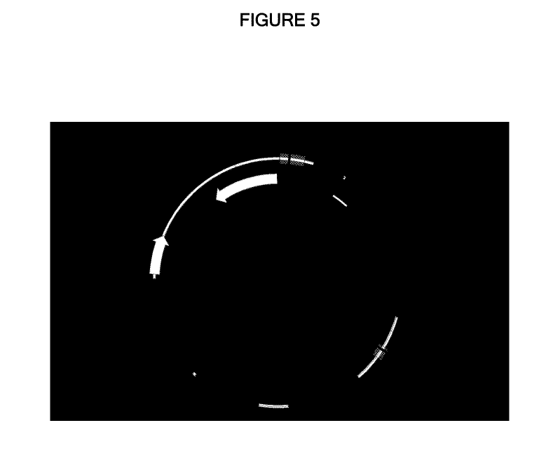

BamHI-SacI fragments containing the synthetic genes encoding different fusion proteins were cloned into BamHI/SacI-digested pNOV1457 to form binary vectors pBSC18238 (FIG. 2, Rhodobacter capsulatus CrtI), pSBC18239 (FIG. 3, Gloeobacter violaceous CrtI), pBSC18240 (FIG. 4, Neurospora CrtI) and pBSC18241 (FIG. 5, Synechococcus PDS V403G mutant) for plant transformation. In these vectors, different phytoene dehydragenase/dasaturase coding sequences were placed under control of the CMP promoter (Stavolone et al., 2003, Cestrum yellow leaf curling virus (CmYLCV) promoter: a new strong constitutive promoter for heterologous gene expression in a wide variety of crops. Plant Mol Biol. 53:663-73). These vectors were transformed into disarmed Agrobacterium strain EHA101 for tobacco and soybean transformation and LBA4404(pSB1) for maize transformation.

### Example 3

### Tobacco Transformation

Young tobacco leaf explants were transformed with norflurazon, a PDS inhibitor, as a selection agent with Agroabcteriem EHA101 carrying binary vector 18238, 18239, 18240 and 18241, respectively. Transformation was carried out using variety SR1 as described (Chilton and Que, 2003, Plant Physiol. 133:956-65) except for using norflurazon as the selection agent. Green calli after norflurazon selection were moved to regeneration medium to produce shoots. Regenerated shoots were rooted and subjected to Taqman analysis for the presence of transgene sequences and their copy numbers. Almost all regenerated shoots contained transgenes. About 60% of transgenic plants contained single copy T-DNA insertion. 18238 and 18239 had high transformation efficiency (>10%), 18241 had lower efficiency (5%), 18240 had lowest transformation efficiency (<1%). The results indicates that all CrtI genes function as selectable markers when fused with chloroplast transit peptide coding sequences and suggest that active enzymes are being transported into chloroplast to confer resistance to herbicide norflurazon, while the genes from Rhodobacter capsulatus and Gloeobacter violaceous performed the best.

### Example 4

### Soybean Transformation

Soybean immature seed explants were transformed with norflurazon as selection agent with Agroabcteriem EHA101 carrying binary vector 18238, 18239, 18240 and 18241, respectively. Transformation was carried out using soybean varieties Jack or Williams 82 as described (Hwang et al., US patent application 2008229447 and Que et al., WO08112267) except for using norflurazon as selection. 4-5 uM of norflurazon was used in regeneration media and 2 to 4 uM was used in elongation media. Regenerate plants were analyzed by Taqman for the presence of transgene sequences and their copy numbers. Transgenic soybean plants were recovered from all 4 vectors. However, similar to tobacco transformation, transformation experiments with vectors carrying Crt/genes from Rhodobacter capsulatus and Gloeobacter violaceous produced more events, suggesting that these bacterial genes confer higher resistance to herbicide norflurazon. Transgenic T0 soybean plants were grown to maturity in greenhouse and allowed to produce transgenic T1 seeds. These seeds were used for evaluating inheritance of transgene and herbicide tolerance in soybean.

### Example 5

### Herbicide Tolerance of Transgenic Soybean Plants Containing CrtI Genes

T1 seeds of transgenic plants from vectors 18238 and 18239 were germinated in soil and grown in the greenhouse to 2-leaf (V2) stage. Plants were sampled for Taqman analysis for the presence of the transgene. Plants were also sprayed with Solicam® herbicide (active ingredient norflurazon) at 1×, 2×, and 4× field application rate (1× rate is 1 lbs./acre). Plants were scored for resistance after 1 week of spraying. Solicam® resistance phenotype correlated with the presence of transgene (Table 2).

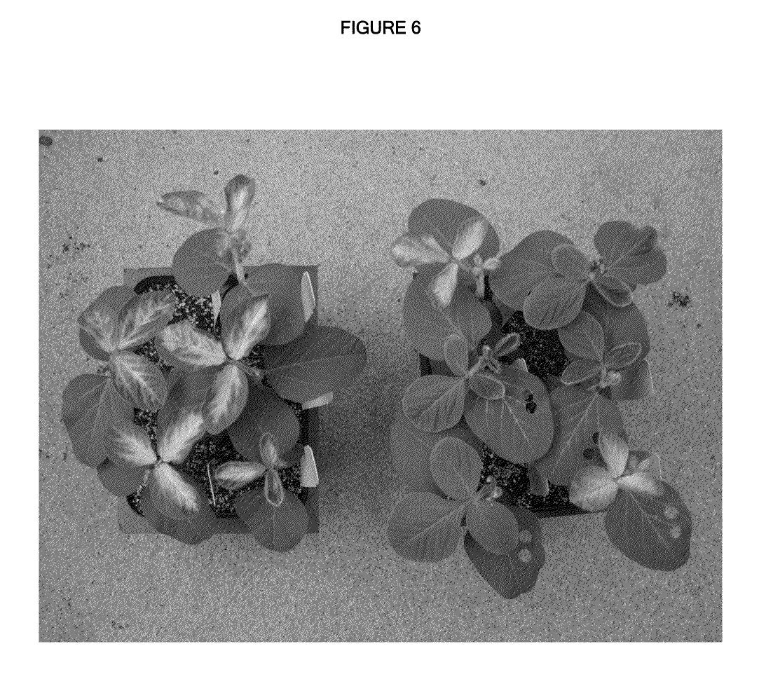

New growth from the resistant plants showed green color, whereas susceptible plants showed bleached phenotype (FIG. 6). The bleached plants eventually died, whereas the resistant soybean plants set seeds normally. The results demonstrate that Solicam® (or norflurazon)-tolerant soybean plants are generated through overexpression of either Rhodobacter capsulatus or Gloeobacter violaceous crtI fusion genes.

### Example 6

### Stacking of CrtI Gene with Trait Genes Including Other Herbicide Tolerance Genes

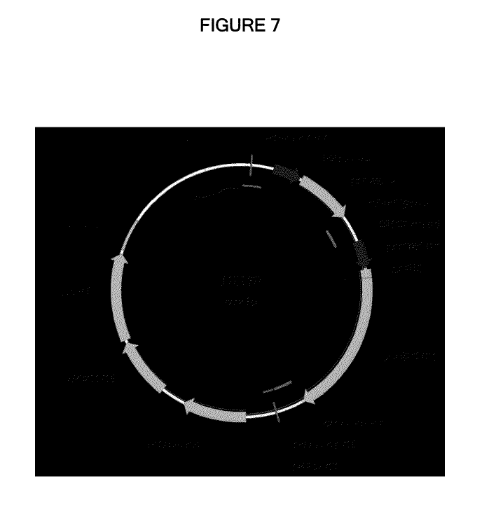

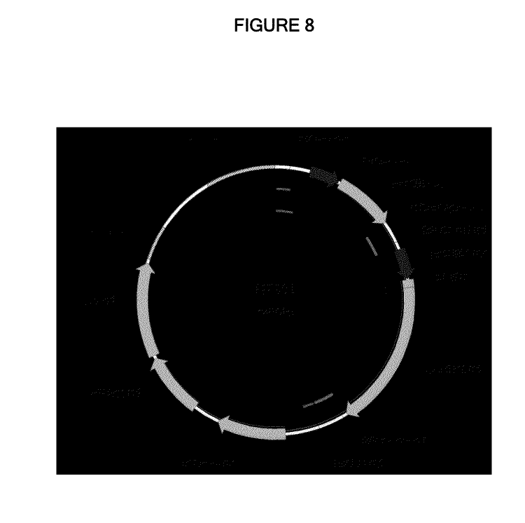

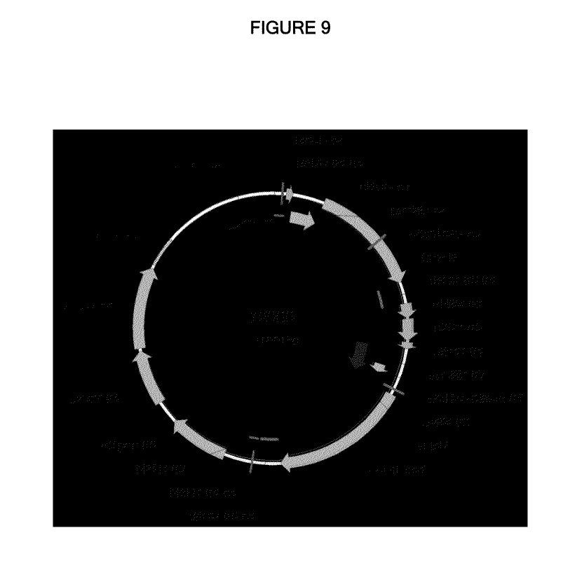

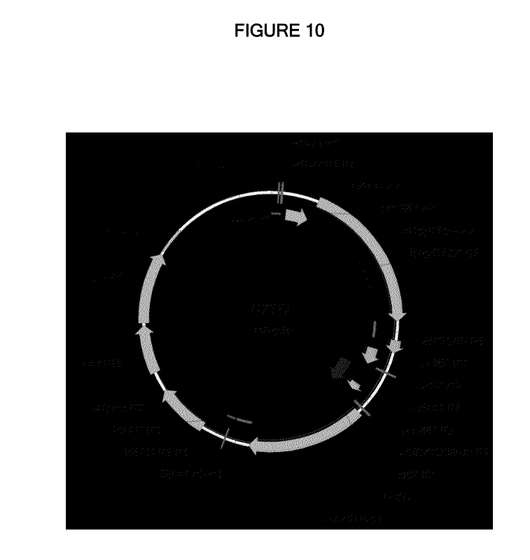

In order to test expression of stacked genes when phytoene desaturease is used as selectable marker, we constructed several vectors with Anemonia majano cyan fluorescent protein gene AmCyan and herbicide resistance (ADSS) or detoxification (cytochrome P450) genes as examples. Binary transformation vector 18340 (FIG. 7) contains Gloeobacter CrtI expression cassette linked to an AmCyan expression cassette. 18340 was derived from insertion of prCMP-AmCyan-tNOS cassette into 18239. Similarly, binary transformation vector 18341 (FIG. 8) contains Rhodobacter CrtI and AmCyan expression cassettes and was constructed by inserting the prCMP-AmCyan-tNOS cassette into 18238. 18900 (FIG. 9) is a binary vector containing Rhodobacter CrtI expression cassette linked with an Arabidopsis adenylosuccinate synthase (ADSS) Q289N mutant expression cassette under the control of prCMP promoter. 18932 (FIG. 10) is a binary vector containing Rhodobacter CrtI expression cassette and an expression cassette for rice cytochrome P450 monooxygenase gene (Genbank accession # OSJNBb0048A17.18) under the control of prCMP promoter. Likewise, other herbicide tolerance genes such as EPSPS and ALS genes can be inserted into CrtI vector 18238 and 18239 to form transformation vectors for generating events resistant to two or more herbicides and other trait genes such as insect resistance, increased yield, enhanced stress tolerance, and altered grain composition.

Binary vectors 18340, 8341, 18900 and 18932 were transformed into soybean plants using norfurazon as selection agent. Transgenic plants derived from 18340 and 18341 expressed fluorescent protein gene AmCyan normally. 18900 transformants were resistant to herbicidal compound hydantocidin. 18932 transformants were evaluated for their resistance against HPPD herbicide mesotrione.

### Example 7

### Maize Transformation

18238 and 18239 are also used as selectable marker cassettes in maize transformation. 18238 and 18239 are transformed into LBA4404(pSB1) Agrobacterium strain, respectively. LBA4404(pSB1, 18238) and LBA4404 (pSB1,18239) are used to infect maize immature embryos as described except using norflurazon as selection agent under light (Negrotto et al., 2000, Plant Cell Reports, 19:798-803; Li et al 2003, Plant Physiol. 133:736-747). Green calli are transferred to regeneration media for recovering transgenic plants. Regenerated plants are subjected to Taqman assay for detecting presence of transgenes in plants.

TABLE 2
Spray of Transgenic Soybean Plants with Commercial Herbicide Solicam ®

| | | | | | | | # of | # of | |
|---|---|---|---|---|---|---|---|---|---|
| | | | T0 | | | T1 | Solicam- | Solicam ®- | |
| | | | transgene | # of | # of | Taqman | resistant | sensitive | |
| Binary | | T0 Plant | copy | T1 seeds | germinated | copy | Green | bleached | |
| vector | Variety | ID | number | planted | seedlings | number | Seedlings | seedlings | Note |
| 18238 | Jack | SYHT0937 | 2 | 12 | 12 | 1^{~}3 | 12 | 0 | Transgene |
| | | 04A006A | | | | | | | inheritable |
| 18238 | Jack | SYHT0938 | 3 | 12 | 12 | 1^{~}4 | 12 | 0 | Transgene |
| | | 06A001A | | | | | | | inheritable |
| 18238 | Jack | SYHT0938 | 1 | 12 | 11 | 0 | 0 | 11 | T0 Chimera |
| | | 16A002A | | | | | | | |
| 18239 | Jack | SYHT0937 | 0 | 12 | 12 | 0 | 0 | 12 | T0 escape |
| | | 05C004A | | | | | | | |
| 18239 | Jack | SYHT0937 | 2 | 12 | 11 | 2^{~}5 | 8 | 3 | Transgene |
| | | 05C007A | | | | | | | inheritable |
| 18239 | Jack | SYHT0938 | 3 | 12 | 2 | 0^{~}1 | 0 | 2 | Transgene |
| | | 17A059A | | | | | | | inheritable |

1. An isolated DNA molecule encoding a modified PDS enzyme wherein said enzyme comprises an amino acid sequence selected from the group consisting of: SEQ ID NO. 1, SEQ ID NO. 2, SEQ ID NO. 3 and SEQ ID NO. 4.

2. The isolated DNA molecule of claim 1, wherein said DNA molecule comprises a polynucleotide sequence selected from the group consisting of: SEQ ID NO. 5, SEQ ID NO. 6, SEQ ID NO. 7 and SEQ ID NO. 8.

3. A DNA construct comprising a promoter that functions in plant cells operably linked to a fusion protein, said fusion protein comprising a chloroplast transit peptide fused to the isolated DNA molecule of claim 1.

4. The DNA construct of claim 3, wherein said chloroplast transit peptide is SEQ ID NO. 9.

5. The DNA construct of claim 3, comprising a DNA molecule selected from the group consisting of SEQ ID NO. 5, SEQ ID NO. 6, SEQ ID NO. 7 and SEQ ID NO. 8.

6. A method of preparing a PDS inhibitor tolerant plant comprising the steps of:
a. contacting a recipient plant cell with the DNA construct of claim 3, wherein said DNA construct is incorporated into the genome of the recipient plant cell;
b. regenerating the recipient plant cell into a plant; and
c. applying an effective dose of a carotenoid biosynthesis inhibitor to the plant, wherein the plant displays a PDS inhibitor tolerant phenotype.

7. The method of claim 6, wherein the PDS inhibitor is selected from the group consisting of norflurazon, fluridone, diflufenican, flurtamone, flurochloride, picolinafen, LS-80707, SAN 380H, RH1965, and Ref.31.

8. A PDS inhibitor tolerant plant and progeny thereof comprising the DNA construct of claim 3.

9. The plant of claim 8 wherein the plant is a soybean plant.

10. The plant of claim 8 wherein the plant is a corn plant.

11. A method of controlling weeds in a field of PDS inhibitor tolerant crop plants comprising applying to said field of PDS inhibitor tolerant crop plants an effective dose of a PDS inhibitor containing herbicide, wherein said PDS inhibitor tolerant crop plant contains a DNA construct comprising a promoter that functions in plant cells operably linked to a DNA molecule that encodes a chloroplast transit peptide linked to the isolated DNA molecule of claim 1.

12. The method of claim 11, wherein the PDS inhibitor contained within the herbicide is selected from the group consisting of norflurazon, fluridone, diflufenican, flurtamone, flurochloride, picolinafen, LS-80707, SAN 380H, RH1965, and Ref.31.

13. A method of plant transformation wherein the selectable marker is an enzyme encoded by the isolated DNA molecule of claim 1 and the selection agent is a PDS inhibitor herbicide.


SEQUENCE LISTING (10 sequences)

SEQ ID NO: 1 - protein, 524 aa, Rhodobacter capsulatus crtI
MSKNTEGMGRAVVIGAGLGGLAAAMRLGAKGYKVTVVDRLDRPGGRGSSITKGGHRFDLGPTIVTVPDRLRELWADCGRDFDKDVSLVPMEPFYTIDFPDGEKYTAYGDDAKVKAEVARISPGDVEGFRHFMWDAKARYEFGYENLGRKPMSKLWDLIKVLPTFGWLRADRSVYGHAKKMVKDDHLRFALSFHPLFIGGDPFHVTSMYILVSQLEKKFGVHYAIGGVQAIADAMAKVITDQGGEMRLNTEVDEILVSRDGKATGIRLMDGTELPAQVVVSNADAGHTYKRLLRNRDRWRWTDEKLDKKRWSMGLFVWYFGTKGTAKMWKDVGHHTVVVGPRYKEHVQDIFIKGELAEDMSLYVHRPSVTDPTAAPKGDDTFYVLSPVPNLGFDNGVDWSVEAEKYKAKVLKVIEERLLPGVAEKITEEVVFTPETFRDRYLSPLGAGFSLEPRILQSAWFRPHNASEEVDGLYLVGAGTHPGAGVPSVIGSGELVAQMIPDAPKPETPAAAAPKARTPRAKAAQ

SEQ ID NO: 2 - protein, 560 aa, Gloeobacter violaceous crtI
MLRSVIKRRTRKGAALERKKAIVIGSGVGGLSLGIRLQSLGFDTTIFEKLDGPGGRAYVRRAEGFTFDMGPTVITVPHFIEELFALERDQPALDKPDFPAAVLDENRRITSGVSGGPATSKYVQIIPILPFYRIYFDDGTHFDYDGDPERTREQIRAIAPEDLEGYERFHRDARAIFERGFLELGFTYFRDVPSMLRIAPDLVKLDAVRPLFSFVKRYFKSDKLRQVFSFEPLLVGGNPLAVPAIYAMIHFVEKTWGIHFAMGGTGALVRGFVRKFEELGGTMRYGSEVARIEIEGSGKHRRATAVVPADGSRHPADLVASNGDYVNTYLKLIDRVHRPSHPDIRLKLARQSMSLLVVYFGFRSDGLNLDLRHHNIILGPRYEELLKDIFGRKILAEDFSQYLHIPSITDPSLAPPGHHCAYTLVPVPHNGSGLDWSKLADPFVDRVLRFLDERGYIPGLGERLVYKSFITPDYFEHTLNSRLGNGFGIEPVLWQSAYFRPHNKSEDIANFYLVGANTQPGGGTPAVMMSAKMTAREIARDFDIPFDVVDRPQVRGTVAK

SEQ ID NO: 3 - protein, 595 aa, Neurospora crassa crtI
MAETQRPRSAIIVGAGAGGIAVAARLAKAGVDVTVLEKNDFTGGRCSLIHTKAGYRFDQGPSLLLLPGLFRETFEDLGTTLEQEDVELLQCFPNYNIWFSDGKRFSPTTDNATMKVEIEKWEGPDGFRRYLSWLAEGHQHYETSLRHVLHRNFKSILELADPRLVVTLLMALHPFESIWHRAGRYFKTDRMQRVFTFATMYMGMSPFDAPATYSLLQYSELAEGIWYPRGGFHKVLDALVKIGERMGVKYRLNTGVSQVLTDGGKNGKKPKATGVQLENGEVLNADLVVVNADLVYTYNNLLPKEIGGIKKYANKLNNRKASCSSISFYWSLSGMAKELETHNIFLAEEYKESFDAIFERQALPDDPSFYIHVPSRVDPSAAPPDRDAVIALVPVGHLLQNGQPELDWPTLVSKARAGVLATIQARTGLSLSPLITEEIVNTPYTWETKFNLSKGAILGLAHDFFNVLAFRPRTKAQGMDNAYFVGASTHPGTGVPIVLAGAKITAEQILEETFPKNTKVPWTTNEERNSERMRKEMDEKITEEGIIMRSNSSKPGRRGSDAFEGAMEVVNLLSQRAFPLLVALMGVLYFLLFVR

SEQ ID NO: 4 - protein, 474 aa, Synechococcus PCC7942 PDS mutant
MRVAIAGAGLAGLSCAKYLADAGHTPIVYERRDVLGGKVAAWKDEDGDWYETGLHIFFGAYPNMLQLFKELNIEDRLQWKSHSMIFNQPTKPGTYSRFDFPDIPAPINGVAAILSNNDMLTWEEKIKFGLGLLPAMIRGQSYVEEMDQYSWTEWLRKQNIPERVNDEVFIAMAKALNFIDPDEISATVVLTALNRFLQEKKGSMMAFLDGAPPERLCQPIVEHVQARGGDVLLNAPLKEFVLNDDSSVQAFRIAGIKGQEEQLIEADAYVSALPVDPLKLLLPDAWKAMPYFQQLDGLQGVPVINIHLWFDRKLTDIDHLLFSRSPLLSVYADMSNTCREYEDPDRSMLELVFAPAKDWIGRSDEDILAATMAEIEKLFPQHFSGENPARLRKYKIVKTPLSGYKATPGRQQYRPDQASPIANFFLTGDYTMQRYLASMEGAVLSGKLTAQAIIARQDELQRRSSGRPLAASQA

SEQ ID NO: 5 - DNA, 1578 bp, Rhodobacter capsulatus crtI
atgtctaagaacactgagggaatgggaagggctgttgttattggagctggacttggaggacttgctgctgctatgagacttggagctaagggatacaaggttaccgttgtggatagacttgatagaccaggtggaaggggatcttctattaccaagggtggacacagattcgatcttggaccaaccattgtgactgttccagataggcttagagaactttgggctgattgcggaagagatttcgataaggatgtgtcccttgttccaatggaaccattctacaccattgatttcccagatggtgagaagtataccgcttacggtgatgatgctaaggttaaggctgaagtggctagaatttctccaggtgatgttgagggattcaggcatttcatgtgggatgctaaggctagatatgagttcggatacgagaaccttggaagaaagccaatgtctaagctctgggatcttattaaggtgttgccaaccttcggatggcttagagctgatagatctgtttacggacacgccaagaagatggttaaggacgatcatcttaggttcgctttgtctttccacccactcttcattggaggtgatccattccatgtgacctccatgtacattcttgtgtctcagctcgaaaagaagttcggagttcactacgctattggaggtgttcaagctattgctgacgctatggctaaggttatcactgatcagggtggagaaatgagacttaacaccgaggttgacgagattcttgtttccagagatggaaaggctactggaattaggcttatggatggaactgaacttccagctcaggttgtggtttctaacgctgatgctggacatacttacaagaggcttctcagaaacagagataggtggagatggactgatgagaagctcgataagaaaaggtggtctatgggacttttcgtttggtacttcggaactaagggaactgctaagatgtggaaggatgttggacatcataccgttgttgttggaccaaggtacaaagagcacgttcaggacattttcattaagggtgagcttgctgaggatatgtctctttacgttcacaggccatctgttactgatccaactgctgctccaaagggtgatgataccttctacgtgttgtctccagttccaaaccttggattcgataacggtgttgattggtctgttgaggctgagaagtacaaggctaaggtgttgaaggttatcgaagagagacttcttccaggcgttgcagaaaagattaccgaagaggttgtgttcactccagagactttcagggatagatacctttctccacttggtgctggattttctttggagccaaggattcttcaatctgcatggttcagaccacataacgcttcagaagaggttgacggactttatcttgttggagctggaactcatccaggtgctggtgttccatctgttattggttctggtgagcttgttgctcagatgattccagatgctccaaagccagaaactccagctgctgctgctcctaaggctagaactccaagagctaaggctgctcaatgatga

SEQ ID NO: 6 - DNA, 1686 bp, Gloeobacter violaceous crtI
atgctcagatctgtgatcaagagaaggactagaaagggtgctgctcttgaaagaaagaaggccatcgttattggatctggtgttggaggactttctttgggaattaggcttcagtctctcggattcgatacaaccattttcgagaagcttgatggaccaggtggaagggcttatgttagaagggctgagggattcactttcgatatgggaccaaccgttattactgtgccacacttcattgaggaacttttcgctcttgagagagatcaaccagctcttgataagccagatttcccagctgctgttcttgatgagaacagaaggattaccagcggagtttctggtggaccagctacttctaagtacgtgcagatcattccaattctcccattctacaggatctacttcgatgatggaacccacttcgattatgatggtgatccagagagaactagagagcagattagggctattgcaccagaagatcttgaaggatacgagaggttccatagagatgctagggctattttcgagagaggattccttgaacttggcttcacttacttcagggatgtgccatctatgcttaggattgctccagatcttgtgaagcttgatgctgttaggcctcttttcagcttcgtgaaaagatacttcaagtccgataagcttaggcaggttttctctttcgaaccacttcttgttggaggaaacccacttgctgttccagctatctacgctatgattcacttcgttgaaaagacctggggaattcatttcgctatgggaggaactggtgctcttgttagaggattcgtgaggaagttcgaagaacttggaggaactatgagatacggatctgaggttgccagaattgagattgagggatctggaaagcacagaagggctactgctgttgttccagctgatggatctagacatccagctgatcttgttgcttccaacggtgattacgtgaacacctacctcaagcttattgatagagtgcataggccatctcatccagatattaggcttaagcttgctaggcagtctatgtctcttcttgtggtgtatttcggattcaggtctgatggacttaaccttgatcttaggcaccacaacattattcttggacccaggtacgaagaactcctcaaggatattttcggtaggaagatccttgctgaggatttctctcagtaccttcacattccatccattaccgatccatctcttgctccaccaggacatcattgcgcttacacccttgttccagttccacataacggatctggacttgattggtctaagctcgctgatccattcgttgatagggtgctcagattccttgatgagaggggttatattccaggacttggtgagagacttgtgtacaagtccttcatcaccccagattacttcgagcatacccttaactctagactcggaaacggattcggaattgagccagttctttggcaatctgcttacttcaggccacataacaagtctgaggatatcgctaacttctaccttgttggagctaacactcaaccaggtggaggaactccagctgttatgatgtctgctaagatgaccgctagagagattgctagggatttcgatatcccattcgatgttgttgataggccacaagttagaggaactgttgctaagtgatga

SEQ ID NO: 7 - DNA, 1791 bp, Neurospora crassa crtI
atggctgagactcaaagaccaaggtccgctattattgtgggagctggtgctggtggaattgctgttgctgctagacttgctaaggctggtgttgatgttactgtgctcgagaagaacgatttcactggtggaaggtgctcacttattcataccaaggctggatacagattcgatcagggacctagtcttttgcttcttccaggacttttcagagagactttcgaggatcttggaactactcttgagcaagaggatgttgagcttcttcaatgcttccccaactacaacatttggttctccgatggaaagagattctctccaaccactgataacgctaccatgaaggtggaaattgaaaagtgggaaggaccagatggattcagaagatacctttcttggcttgctgaaggacatcaacactacgagacttctcttaggcatgttctccacaggaacttcaagtctattctcgagcttgctgatccaagacttgttgtgacccttcttatggcacttcatccattcgagtctatttggcatagagctggcagatacttcaagactgataggatgcaaagggttttcactttcgccactatgtacatgggaatgtccccattcgatgctccagctacctattctttgcttcagtactctgaacttgctgagggaatttggtatccaaggggaggattccataaggttctcgatgctctcgttaagattggagagaggatgggagttaagtacaggcttaacaccggtgtttctcaagttcttaccgatggtggaaagaacggaaagaagccaaaggctactggcgttcaacttgaaaacggtgaggtgttgaacgctgatcttgttgttgtgaacgctgatttggtgtacacctacaacaacttgctcccaaaagagattggaggcatcaagaagtacgctaacaagctcaacaacaggaaggcttcttgctcctctatttccttctactggtccctttcaggaatggctaaagagcttgagactcacaacattttcttggccgaagagtacaaagaatccttcgatgctattttcgagagacaggctttgccagatgatccatccttctacattcatgtgccatccagagttgatccatctgctgctccaccagatagagatgctgtgattgctcttgttccagttggacaccttcttcaaaacggacaaccagaacttgattggccaacccttgtttctaaggctagagctggtgttcttgctactattcaagctaggaccggactttctctttctccactcatcaccgaagagattgtgaacactccatacacttgggagactaagttcaacctttccaagggtgctattcttggacttgctcacgatttcttcaacgtgttggctttcagaccaagaactaaggctcagggaatggataacgcttatttcgttggagcttctactcatccaggtactggtgttccaattgtgcttgctggtgctaagattactgctgagcagattcttgaagagactttccccaagaacacaaaagttccttggactaccaacgaagaaagaaactctgagaggatgagaaaagagatggacgagaagattaccgaagagggaattatcatgaggtctaactcttctaagccaggaagaagaggatctgacgctttcgaaggtgctatggaagttgtgaaccttttgtctcaaagggctttcccacttcttgttgctcttatgggagtgctttacttcttgctgttcgtgaggtgatga

SEQ ID NO: 8 - DNA, 1428 bp, Synechococcus PCC7942 PDS mutant
atgagagtggctatcgctggtgctggacttgctggtctttcttgcgctaagtaccttgctgatgctggacatactccaattgtgtacgagagaagggatgttcttggaggaaaggttgcagcttggaaggatgaagatggtgattggtacgagactggactccatattttcttcggagcctacccaaacatgcttcagcttttcaaagagctgaacatcgaggatagacttcagtggaagtcccactccatgattttcaaccagccaactaagccaggaacttactccagattcgacttcccagatattccagctcctattaacggtgtggctgctattctttccaacaacgacatgcttacttgggaagagaagattaagttcggacttggacttcttccagctatgattaggggacagtcctacgttgaagagatggatcaatactcttggactgagtggcttaggaagcaaaacattccagagagagtgaacgacgaggttttcattgctatggctaaggctctcaacttcattgatccagatgagatttctgctaccgttgttcttactgctctcaacagattcctccaagagaagaaaggatctatgatggctttccttgatggtgctccaccagaaagactttgccaacctattgtggagcatgttcaagctagaggtggagatgttcttcttaacgccccactcaaagagttcgttctcaacgacgattcttctgttcaggctttcaggattgctggtattaagggacaagaggaacagcttattgaggctgatgcttacgtttcagctttgccagttgatccacttaagttgcttttgccagatgcttggaaggctatgccatacttccaacagcttgatggacttcaaggtgtgccagtgattaacattcacctctggttcgataggaagctcaccgatattgatcaccttttgttctctaggtccccacttctttctgtttacgccgatatgtctaacacttgcagagagtatgaagatccagacagatctatgcttgagcttgttttcgctccagctaaggattggattggaaggtccgatgaggatattcttgctgctactatggctgagatcgagaagcttttcccacaacatttctctggtgagaacccagctagacttaggaagtacaagatcgttaagaccccactttctggatacaaggctactccaggaaggcaacagtatagaccagatcaggcttctccaatcgctaacttcttcctcactggtgattacactatgcagagatacctcgcttctatggaaggtgctgtgctttctggaaagcttactgctcaggctattattgctaggcaagacgagcttcagagaagatcttctggaaggccacttgctgcttcacaagcttaatga

SEQ ID NO: 9 - protein, 65 aa, Cyanophora paradoxa xFNR
MAFVASVPVFANASGLKTEAKVCQKPALKNSFFRGEEVTSRSFFASQAVSAKPATTGEVDTTIRA

SEQ ID NO: 10 - DNA, 195 bp, Cyanophora paradoxa
atggctttcgttgcttcagttccagttttcgctaacgcttctggacttaagactgaggctaaggtttgccaaaagccagctcttaagaactcattcttcaggggagaggaagttacttctagaagtttcttcgcatctcaagctgtttctgctaagccagctactactggtgaagtggataccactattagggct